(19) United States
(12) Patent Application Publication
(10) Pub. No.: US 2003/0045568 A1
(43) Pub. Date: Mar. 6, 2003

(54) Treating hepatitis C viral infections with thiosemicarbazone compounds

(76) Inventors: Sergio Altamura, Rome (IT); Uwe Koch, Albano Laziale (IT)

Correspondence Address: MERCK AND CO INC, P O BOX 2000, RAHWAY, NJ, 070650907

(21) Appl. No.: 10/125,940

(22) Filed: Apr. 19, 2002

Related U.S. Application Data
(60) Provisional application No. 60/285,104, filed on Apr. 20, 2001.

(51) Int. Cl. A61K 31/381; A61K 31/34; C07D 333/20; C07D 307/02

(52) U.S. Cl. 514/438; 514/471; 549/75; 549/494

(57) ABSTRACT

Thiosemicarbazone compounds of formula:

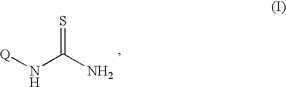

are useful for treating infection by the hepatitis C virus, treating hepatitis C or a related condition, delaying the onset of hepatitis C or a related condition, preventing hepatitis C or a related condition, and inhibiting replication of the hepatitis C virus. In the formula Q is

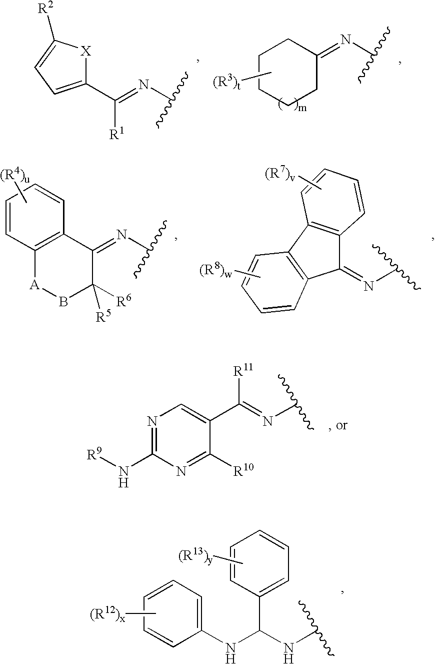

wherein R¹, R², R³, R⁴, R⁵, R⁶, R⁷, R⁸, R⁹, R¹⁰, R¹¹, R¹², and R¹³, are substituents as defined herein, and m, t, u, v, w, x and y are integers as defined herein.

### CROSS REFERENCE TO RELATED APPLICATIONS

This application claims the benefit of U.S. Provisional Application No. 60/285,104 filed Apr. 20, 2001, the disclosure of which is hereby incorporated by reference in its entirety.

### FIELD OF THE INVENTION

The present invention is directed to the use of thiosemicarbazone compounds to treat infection by the hepatitis C virus, treat or delay the onset of hepatitis C, and inhibit replication of the hepatitis C virus.

### BACKGROUND OF THE INVENTION

The hepatitis C virus (HCV) is the major causative agent of parenterally-transmitted and sporadic non-A, non-B hepatitis. It is believed that about 3 percent of the world's population and 2 percent of the U.S. population have been infected with this agent at some time. Exposure to HCV can result in an overt acute disease, but in most cases the virus establishes a chronic infection that causes liver inflammation and slowly progresses into liver failure and cirrhosis, as described for example in Iwarson, FEMS Microbiol. Rev. 1994, 14: 201-204. Epidemiological surveys have also indicated an important role of HCV in the pathogenesis of hepatocellular carcinoma, as described for example in Kew, FEMS Microbiol. Rev. 1994, 14: 211-220. No vaccine or established therapy currently exists, although partial success has been achieved in a minority of cases by treatment with recombinant interferon-alpha, either alone or in combination with ribavirin. There is accordingly a need for the development of alternative anti-HCV therapies.

The following references provide technical background for the present invention:

D. J. Bauer, Brit. Med. Bull. 1985, 41: 309-314 discloses that certain para-substituted benzaldehyde thiosemicarbazones (e.g., p-aminobenzaldehyde thiosemicarbazone and p-methoxybenzaldehyde thiosemicarbazone) are effective against the vaccinia virus (i.e., the cowpox virus), and also discloses that the 1-methyl isatin beta-thiosemicarbazone (M-IBT) and 1-ethyl isatin beta-thiosemicarbazone (E-IBT) are active against the vaccinia virus and the smallpox virus. D. J. Bauer, Ann. N.Y. Acad. Sci., 1965, 130: 110-117 contains a similar disclosure.

U.S. Pat. No. 4,927,843 (Teitz, 1990) discloses that certain isatin thiosemicarbazone derivatives are useful against viruses of the Retroviridae family. The patent specifically discloses 1-methylisatin-β-4′:4′-diethylthiosemicarbazone (M-IBDET), 1-allylisatin-β-4′:4′-dimethylthiosemicarbazone (A-IBDMT), and 1-allylisatin-β-4′:4′-diethylthiosemicarbazone (A-IBDET). Teitz et al., Antiviral Res. 1994, 24: 305-314, discloses inhibition of HIV by M-IBDET and by 1-allylisatin-β-4′:4′-diallylthiosemicarbazone (A-IBDAT).

Ronen et al., Antimicrob. Agents and Chemotherapy 1987, 31: 1798-1802 analyzes the mode of inhibition of Moloney leukemia virus production by M-IBDET. Teitz et al., Chemotherapy 1994, 40: 195-200, discloses that A-IBDAT is also an effective inhibitor of the Money leukemia virus.

Peloquin et al., Phamacotherapy 1996, 16: 735-741, presents a pharmacokinetic evaluation of thiacetazone, which is a known antimycobacterial agent useful for treating tuberculosis.

Finch et al., Biochemical Pharmacology 2000, 59: 983-991 discloses that 3-aminopyridine-2-carboxaldehyde thiosemicarbazone is a human ribonucleotide reductase inhibitor useful for treating cancer.

U.S. Pat. No. 5,098,462 (Anderson et al., 1992) and U.S. Pat. No. 5,098,466 (Anderson et al., 1992) each disclose certain (aryl- and aza-aryl-aldehyde and ketone)-4-(aryl- or aza-aryl-)thiosemicarbarzones which are said to be useful for the control of weeds.

U.S. Pat. No. 5,281,597 (McCall et al; 1994) discloses certain heterocyclic and aromatic thiosemicarbazones which are said to be useful for the treatment of filariasis.

U.S. Pat. No. 5,344,842 (Missbach, 1994) discloses certain (4-oxo-thiazolidin-2-ylidene)-thiosemicarbazones which are said to be useful for the treatment of diseases of the rheumatoid type. Similarly, U.S. Pat. No. 5,641,776 (Missbach, 1997) discloses certain (4-oxo-[1,3]thiazinan-2-ylidene)-thiosemicarbazones which are said to be useful for the treatment of diseases of the rheumatoid type.

U.S. Pat. No. 5,376,685 (Stanek et al., 1994) discloses certain amidine and amide substituted aryl and aza-aryl-hydrazones which are said to be useful as SAMDC inhibitors, wherein SAMDC is an enzyme that plays an important role in polyamine synthesis occurring in the cells of mammals.

U.S. Pat. No. 5,942,527 (Kadaba et al., 1999) discloses certain thiosemicarbazones derived from certain pyridyl ketones which are said to be useful for the treatment of stroke and other neurological disorders.

### SUMMARY OF TEE INVENTION

The present invention is directed to the use of certain thiosemicarbazone compounds for the treatment of infection by the hepatitis C virus, the treatment of hepatitis C, the delay in the onset of hepatitis C, prevention of hepatitis C, and the inhibition of the hepatitis C virus. More particularly, the present invention is a method for treating infection by the hepatitis C virus, treating hepatitis C or a related condition, delaying the onset of hepatitis C or a related condition, preventing hepatitis C or a related condition, or inhibiting replication of the hepatitis C virus, which comprises administering to a subject in need thereof an effective amount of a compound of Formula (I):

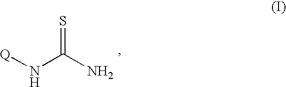

or a pharmaceutically acceptable salt thereof;

wherein Q is selected from the group consisting of:

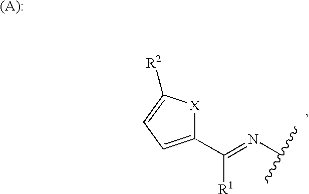

wherein

X is S or O;

R¹ is —H or —C₁₋₄ alkyl; and

R² is:

(1) —C₁₋₆ alkyl,

(2) —O—C₁₋₆ alkyl,

(3) —C₃₋₈ cycloalkyl,

(4) —O—C₃₋₈ cycloalkyl,

(5) —Si(R^{a})_{3,} in which one R^{a} group is phenyl optionally substituted with from 1 to 3 substituents each of which is independently —C₁₋₆ alkyl or —O—C₁₋₆ alkyl; and the other R^{a} groups are independently methyl, ethyl, methoxy, ethoxy, or phenyl optionally substituted with from 1 to 3 substituents each of which is independently —C₁₋₆ alkyl or —O—C₁₋₆ alkyl,

(6) —Cl or —Br,

(7) phenyl, optionally substituted with from 1 to 3 substituents each of which is independently —C₁₋₆ alkyl, —O—C₁₋₆ alkyl, or —C₃₋₈ cycloalkyl, or

(8) —(C₁₋₆ alkyl)-phenyl, in which the phenyl is optionally substituted with from 1 to 3 substituents each of which is independently —C₁₋₆ alkyl, —O—C₁₋₆ alkyl, or —C₃₋₈ cycloalkyl;

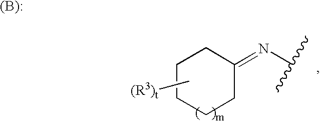

wherein

each R³ is independently a —C₁₋₆ alkyl group;

m is an integer from 2 to 9; and

t is zero, 1, 2, 3, or 4;

(C):

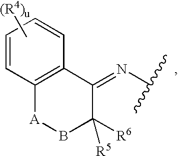

wherein

A is absent or —C(R^{b}R^{c})—;

B is —C(R^{d}R^{e})— or —NR^{f}—;

each R⁴ is independently:

(1) —C₁₋₆ alkyl,

(2) —O—C₁₋₆ alkyl,

(3) —C₃₋₈ cycloalkyl,

(4) —O—C₃₋₈ cycloalkyl,

(5) —Cl or —Br,

(6) phenyl, optionally substituted with from 1 to 3 substituents each of which is independently —C₁₋₆ alkyl, —O—C₁₋₆ alkyl, or —C₃₋₈ cycloalkyl, or

(7) —(C₁₋₆ alkyl)-phenyl, in which the phenyl is optionally substituted with from 1 to 3 substituents each of which is independently —C₁₋₆ alkyl, —O—C₁₋₆ alkyl, or —C₃₋₈ cycloalkyl;

R⁵ and R⁶ are each independently —H or —C₁₋₄ alkyl; or R⁵ and R⁶ taken together form oxo;

u is an integer equal to zero, 1, or 2;

R^{b} and R^{c} are each independently —H or —C₁₋₄ alkyl;

R^{d} and R^{e} are each independently —H or —C₁₋₄ alkyl; and

R^{f} is —H or —C₁₋₄ alkyl;

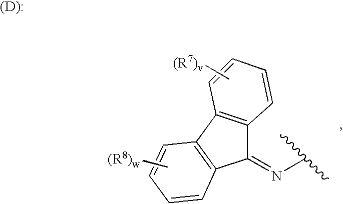

wherein each R⁷ and each R⁸ is independently:

(1) —C₁₋₆ alkyl,

(2) —O—C₁₋₆ alkyl,

(3) —C₃₋₈ cycloalkyl,

(4) —O—C₃₋₈ cycloalkyl,

(5) —Cl or —Br,

(6) phenyl, optionally substituted with from 1 to 3 substituents each of which is independently —C₁₋₆ alkyl, —O—C₁₋₆ alkyl, or —C₃₋₈ cycloalkyl, or

(7) —(C₁₋₆ alkyl)-phenyl, in which the phenyl is optionally substituted with from 1 to 3 substituents each of which is independently —C₁₋₆ alkyl, —O—C₁₋₆ alkyl, or —C₃₋₈ cycloalkyl; and

v and w are each integers independently equal to zero, 1, 2 or 3;

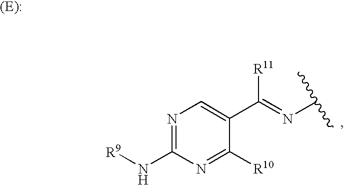

wherein R⁹ is:

(1) —C₁₋₆ alkyl,

(2) —C₃₋₈ cycloalkyl,

(3) phenyl, optionally substituted with from 1 to 3 substituents each of which is independently —C₁₋₆ alkyl, —O—C₁₋₆ alkyl, or —C₃₋₈ cycloalkyl, or

(4) —(C₁₋₆ alkyl)-phenyl, in which the phenyl is optionally substituted with from 1 to 3 substituents each of which is independently —C₁₋₆ alkyl, —O—C₁₋₆ alkyl, or —C₃₋₈ cycloalkyl;

R¹⁰ is —H or —C₁₋₄ alkyl; and

R¹¹ is —H or —C₁₋₄ alkyl; and

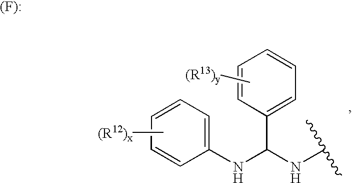

wherein each R¹² and each R¹³ is independently:

(1) —C₁₋₆ alkyl,

(2) —O—C₁₋₆ alkyl,

(3) —C₃₋₈ cycloalkyl,

(4) —O—C₃₋₈ cycloalkyl,

(5) —Cl or —Br,

(6) phenyl, optionally substituted with from 1 to 3 substituents each of which is independently —C₁₋₆ alkyl, —O—C₁₋₆ alkyl, or —C₃₋₈ cycloalkyl, or

(7) —(C₁₋₆ alkyl)-phenyl, in which the phenyl is optionally substituted with from 1 to 3 substituents each of which is independently —C₁₋₆ alkyl, —O—C₁₋₆ alkyl, or —C₃₋₈ cycloalkyl; and

x and y are each integers independently equal to zero, 1, 2 or 3.

Embodiments, aspects and features of the present invention are either further described in or will be apparent from the ensuing description, examples, and appended claims.

### DETAILED DESCRIPTION OF THE INVENTION

A first embodiment of the present invention is a method for treating infection by the hepatitis C virus, treating hepatitis C or a related condition, delaying the onset of hepatitis C or a related condition, preventing hepatitis C or a related condition, or inhibiting replication of the hepatitis C virus, which comprises administering to a subject in need thereof an effective amount of a compound of Formula (II):

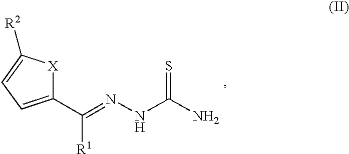

or a pharmaceutically acceptable salt thereof;

wherein X is S or O;

R¹ is —H, methyl, or ethyl; and

R² is:

(1) methyl,

(2) ethyl,

(3) methoxy,

(4) ethoxy,

(5) —C₅₋₆ cycloalkyl,

(6) —Si(R^{a})₃, in which one R^{a} group is phenyl and the other R^{a} groups are independently methyl, ethyl, or phenyl,

(7) —Cl,

(8) —Br,

(9) phenyl, or

(10) —CH₂-phenyl.

An aspect of the first embodiment is a method as set forth above wherein in the compound of Formula (II) or a pharmaceutically acceptable salt thereof:

X is S or O;

R¹ is —H, methyl, or ethyl; and

R² is:

(1) methyl,

(2) ethyl,

(3) methoxy,

(4) ethoxy,

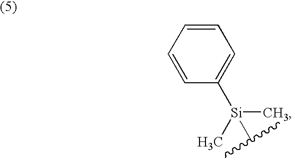

(6) —Cl, or

(7) —Br.

Exemplary of the compounds employed in the method of the present invention as set forth in the first embodiment is a compound selected from the group consisting of:

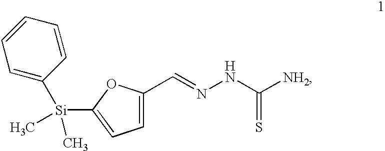

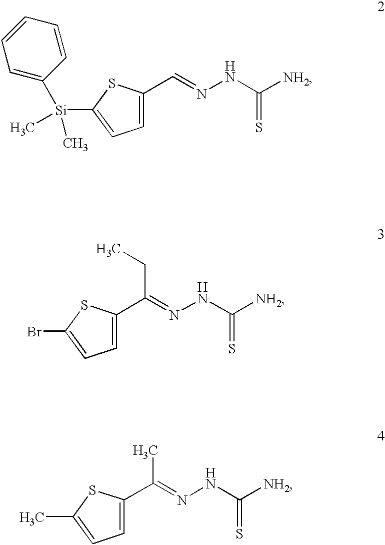

and pharmaceutically acceptable salts thereof.

A second embodiment of the present invention is a method for treating infection by the hepatitis C virus, treating hepatitis C or a related condition, delaying the onset of hepatitis C or a related condition, preventing hepatitis C or a related condition, or inhibiting replication of the hepatitis C virus, which comprises administering to a subject in need thereof an effective amount of a compound of Formula (III):

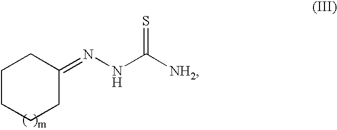

wherein m is an integer from 5 to 9;

or a pharmaceutically acceptable salt thereof.

Exemplary of the compounds employed in the method of the present invention as set forth in the second embodiment is

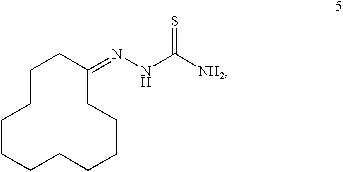

or a pharmaceutically acceptable salt thereof.

A third embodiment of the present invention is a method for treating infection by the hepatitis C virus, treating hepatitis C or a related condition, delaying the onset of hepatitis C or a related condition, preventing hepatitis C or a related condition, or inhibiting replication of the hepatitis C virus, which comprises administering to a subject in need thereof an effective amount of a compound of Formula (IV):

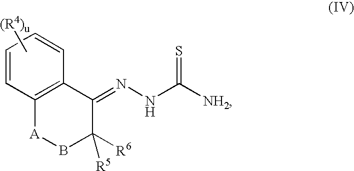

or a pharmaceutically acceptable salt thereof;

wherein:

A is absent or —CH₂—;

B is —CH₂— or —NR^{f}—, with the proviso that when B is —NR^{f}—, A is absent;

each R⁴ is independently:

(1) —C₁₋₄ alkyl,

(2) —O—C₁₋₄ alkyl, or

(3) —Cl or —Br,

R⁵ and R⁶ are both —H; or R⁵ and R⁶ taken together form oxo;

u is zero, 1, or 2; and

R^{f} is —H or methyl.

Exemplary of the compounds employed in the method of the present invention as set forth in the third embodiment is a compound selected from the group consisting of:

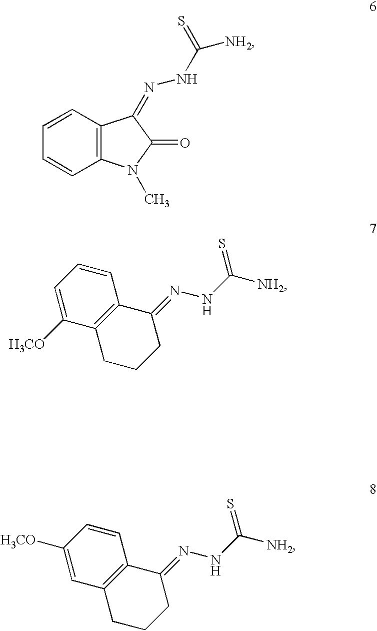

and pharmaceutically acceptable salts thereof.

A fourth embodiment of the present invention is a method for treating infection by the hepatitis C virus, treating hepatitis C or a related condition, delaying the onset of hepatitis C or a related condition, preventing hepatitis C or a related condition, or inhibiting replication of the hepatitis C virus, which comprises administering to a subject in need thereof an effective amount of a compound of Formula (V):

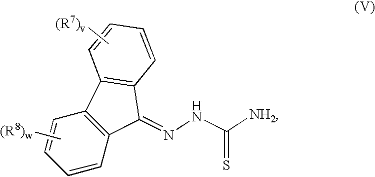

or a pharmaceutically acceptable salt thereof;

wherein each R⁷ and each R⁸ is independently:

(1) —C₁₋₄ alkyl,

(2) —O—C₁₋₄ alkyl, or

(3) —Cl or —Br; and

v and w are each integers independently equal to zero, 1, or 2.

Exemplary of the compounds employed in the method of the present invention as set forth in the fourth embodiment is

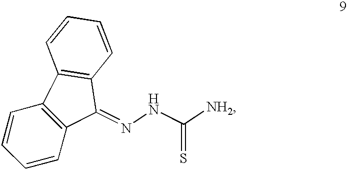

or a pharmaceutically acceptable salt thereof.

A fifth embodiment of the present invention is a method for treating infection by the hepatitis C virus, treating hepatitis C or a related condition, delaying the onset of hepatitis C or a related condition, preventing hepatitis C or a related condition, or inhibiting replication of the hepatitis C virus, which comprises administering to a subject in need thereof an effective amount of a compound of Formula (VI):

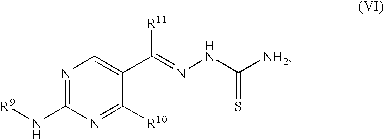

or a pharmaceutically acceptable salt thereof;

wherein R⁹ is —C₆₋₈ cycloalkyl;

R¹⁰ is —H or methyl; and

R¹¹ is —H or methyl.

Exemplary of the compounds employed in the method of the present invention as set forth in the fifth embodiment is

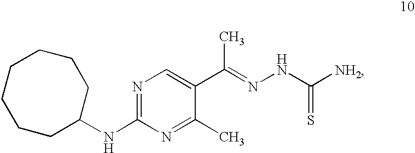

or a pharmaceutically acceptable salt thereof.

A sixth embodiment of the present invention is a method for treating infection by the hepatitis C virus, treating hepatitis C or a related condition, delaying the onset of hepatitis C or a related condition, preventing hepatitis C or a related condition, or inhibiting replication of the hepatitis C virus, which comprises administering to a subject in need thereof an effective amount of a compound of Formula (VII):

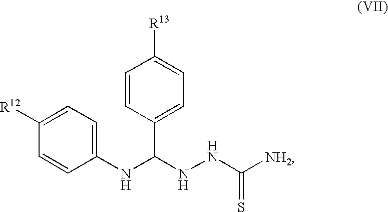

or a pharmaceutically acceptable salt thereof;

wherein R¹² and R¹³ are each independently:

(1) —H,

(2) —C₁₋₆ alkyl,

(3) —O—C₁₋₆ alkyl,

(4) —C₅₋₆ cycloalkyl,

(5) —O—C₅₋₆ cycloalkyl,

(6) —Cl or —Br,

(7) phenyl, optionally substituted with from 1 to 3 substituents each of which is independently —C₁₋₆ alkyl or —O—C₁₋₆ alkyl, or

(8) —(C₁₋₆ alkyl)-phenyl, in which the phenyl is optionally substituted with from 1 to 3 substituents each of which is independently —C₁₋₆ alkyl or —O—C₁₋₆ alkyl.

Exemplary of the compounds employed in the method of the present invention as set forth in the sixth embodiment is

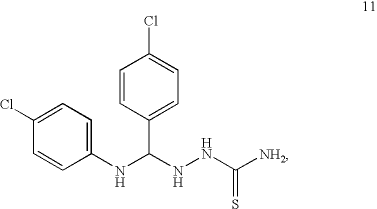

or a pharmaceutically acceptable salt thereof.

The present invention also includes a compound of Formula (I) as defined and described above for use in (a) treating infection by the hepatitis C virus, (b) treating hepatitis C or a related condition, (c) delaying the onset of hepatitis C or a related condition, (d) preventing hepatitis C or a related condition, or (e) inhibiting replication of the hepatitis C virus. The present invention further includes use of a compound of Formula (I) as defined and described above as a medicament for (a) treating infection by the hepatitis C virus, (b) treating hepatitis C or a related condition, (c) delaying the onset of hepatitis C or a related condition, (d) preventing hepatitis C or a related condition, or (e) inhibiting replication of the hepatitis C virus. The present invention also includes use of a compound of Formula (I) as defined and described above in the preparation of a medicament for (a) treating infection by the hepatitis C virus, (b) treating hepatitis C or a related condition, (c) delaying the onset of hepatitis C or a related condition, (d) preventing hepatitis C or a related condition, or (e) inhibiting replication of the hepatitis C virus.

As used herein, the term “C₁₋₆ alkyl” refers to a linear or branched chain alkyl group having from 1 to 6 carbon atoms, and is selected from the hexyl alkyl and pentyl alkyl isomers, n-, iso-, sec- and t-butyl, n- and isopropyl, ethyl and methyl. “C₁₋₄ alkyl” has an analogous definition.

The term “C₃₋₈ cycloalkyl” refers to a cyclic ring selected from cyclopropyl, cyclobutyl, cyclopentyl, cyclohexyl, cycloheptyl, or cyclooctyl. “C₅₋₆ cycloalkyl” has an analogous definition.

The term “substituted” includes mono- and poly-substitution by a named substituent to the extent such single and multiple substitution is chemically allowed and results in a chemically stable compound.

The symbol “ [symbol] ” in front of an open bond in the structural formula of a group marks the point of attachment of the group to the rest of the molecule.

When A is “absent” in Formula (IV), it is understood that B and the ring carbon adjacent to A are directly connected by a single bond. In other words, when A is absent, the compound of Formula (IV) is represented as follows:

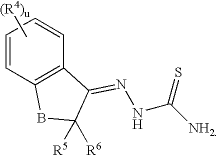

The term “therapeutically effective amount” (or alternatively and more simply “effective amount”) as used herein means that amount of active compound or pharmaceutical agent that elicits the biological or medicinal response in a tissue, system, animal or human that is being sought by a researcher, veterinarian, medical doctor or other clinician, which includes alleviation of the symptoms of the disease being treated and/or the prevention or delay in onset or recurrence of a pathology.

The expression “pharmaceutically acceptable” means that the salt, carrier, diluent or excipient must be compatible with the other ingredients of the formulation and not deleterious to the recipient thereof.

The term “subject” (or alternatively “patient”) as used herein refers to a human or other animal, typically a mammal, who is susceptible to HCV infection and who is the object of treatment, observation or experiment.

The term “administration” and variants thereof (e.g., “administering” a compound) in reference to the present invention mean providing a compound of Formula (I) or a pharmaceutical composition comprising Compound I to the subject or individual in need of treatment. When Compound I is provided in combination with one or more other active agents useful for treating HCV infection or hepatitis C, “administration” and its variants are each understood to include concurrent and time-separated (e.g., alternating) provision of Compound I and other agents.

A “related condition” is a condition which is or can be caused, directly or indirectly, by the hepatitis C virus or with which HCV is associated.

Compounds of Formula (I) may be administered in the form of pharmaceutically acceptable salts. The term “pharmaceutically acceptable salt” refers to all acceptable salts of the compounds of Formula (I) (in the form of water- or oil-soluble or dispersible products) and includes the conventional non-toxic salts formed from inorganic and organic acids or the quaternary ammonium salts formed by reaction with, e.g., alkyl halides. The salt (e.g., hydrochloride salt) can be used as a dosage form for modifying the solubility or hydrolysis characteristics of the compound or can be used in sustained release or pro-drug formulations.

In the methods and uses of the present invention, the compound of Formula (I) may be administered orally, parenterally (including subcutaneous injections, intravenous, intramuscular, intrasternal injection or infusion techniques), by inhalation spray, or rectally, in dosage unit formulations containing conventional non-toxic pharmaceutically-acceptable carriers, adjuvants and vehicles. Accordingly, the present invention includes the methods of treating infection by the hepatitis C virus, treating hepatitis C or a related condition, delaying the onset of hepatitis C or a related condition, preventing hepatitis C or a related condition, or inhibiting replication of the hepatitis C virus, as heretofore described in which Compound I is administered as a pharmaceutical composition comprising Compound I and a pharmaceutically acceptable carrier, adjuvant or vehicle.

These pharmaceutical compositions may be in the form of orally-administrable suspensions or tablets, nasal sprays, sterile injectible preparations, for example, as sterile injectible aqueous or oleagenous suspensions or suppositories.

When administered orally as a suspension, these compositions are prepared according to techniques known in the art of pharmaceutical formulation and may contain microcrystalline cellulose for imparting bulk, alginic acid or sodium alginate as a suspending agent, methylcellulose as a viscosity enhancer, and sweeteners/flavoring agents known in the art. As immediate release tablets, these compositions may contain microcrystalline cellulose, dicalcium phosphate, starch, magnesium stearate and lactose and/or other excipients, binders, extenders, disintegrants, diluents and lubricants known in the art.

When administered by nasal aerosol or inhalation, these compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The injectible solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

When rectally administered in the form of suppositories, these compositions may be prepared by mixing the drug with a suitable non-irritating excipient, such as cocoa butter, synthetic glyceride esters of polyethylene glycols, which are solid at ordinary temperatures, but liquefy and/or dissolve in the rectal cavity to release the drug.

The compounds of this invention can be administered orally to humans in a dosage range of 0.01 to 1000 mg/kg body weight per day in a single dose or in divided doses. One preferred dosage range is 0.1 to 200 mg/kg body weight per day orally in a single dose or in divided doses. Another preferred dosage range is 0.5 to 100 mg/kg body weight per day orally in single or divided doses. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The compounds of Formula (I) employed in the present invention in which Q is the group defined in A, B, C, D and E can be prepared by reacting the corresponding carbonyl compound with thiosemicarbazide in the presence of a suitable solvent (e.g., an alcohol such as methanol or ethanol) and with or without an acid catalyst (e.g., aqueous HCl or aqueous acetic acid) at room temperature or with moderate heating to give the thiosemicarbazone. Illustrative is the preparation of heteroaryl thiosemicarbazones of Formula (II) by reaction of the corresponding ketone or aldehyde VIII with thiosemicarbazide IX:

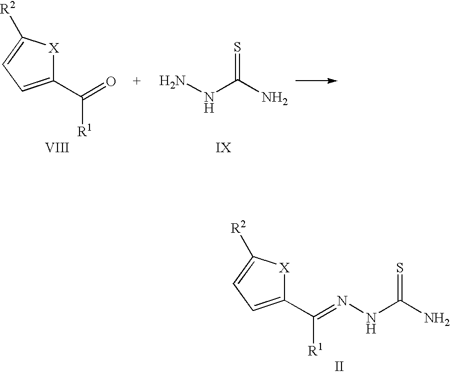

Thiosemicarbazide IX is widely available commercially (see, e.g., suppliers listed in Chem Sources—USA, 2000 edition, Chemical Sources International, Inc., Clemson, S.C.) or can be prepared from hydrazine sulfate and ammonium thiocyanate in the presence of sufficient potassium carbonate to half neutralize the hydrazine sulfate. Many of the starting ketones and aldehydes for preparing thiosemicarbazones I are also commercially available, and the others can be prepared by the person of ordinary skill in the art without undue experimentation by means known in the art. For example, compounds 1 and 2 can be prepared from the commercially available 5-bromothiophene-2-carboxaldehyde or 5-bromo-2-furaldehyde by suitable protection of the aldehyde functionality and subsequent metallation, silylation and deprotection using procedures known in the art. Compounds 3-9 can be prepared from commercially available ketones. Compound 10 can be prepared from the ketone prepared by aminating 4-methylpyrimidine with the alkali metal amide of cyclooctylamine, followed by Friedel-Crafts acylation with an acetyl halide. Alternatively, the ketone required for the preparation of Compound 10 can be obtained through modification of procedures described in Indian J. Chem. Sect. B 1977, 15B: 1129-1132.

Compounds of Formula (I) in which Q is the group defined by F can be prepared by methods known in the art. Compound 11, for example, can be prepared as described in Ismail, Pak. J. Sci. Ind. Res. 1995, 38: 67-70 or El-Deen et al., Arch. Pharmacal Res. 1994, 17: 294-297.

Abbreviations used in the instant specification, particularly in the Examples, include:

DMEM=Dulbecco's modified minimum essential medium (also referred known as Dulbecco's modified eagle medium)

DMSO=dimethylsulfoxide

FCS=fetal calf serum

MS=mass spectrometry

NMR=nuclear magnetic resonance

PBS=phosphate buffered saline

SDS=sodium dodecyl sulfate

SRB=sulphorhodamine B

TCA=trichloroacetic acid

TMB=3,3′,5,5′-tetramethylbenzidine

The following examples serve only to illustrate the invention and its practice. The examples are not to be construed as limitations on the scope or spirit of the invention.

### EXAMPLE 1

### RHEPLISA Assay—A Cell-Based Assay for Measuring HCV Replication

A cell-based assay for measuring the HCV replication (RHEPLISA) was developed in a 96-well microplate format. The assay utilizes Huh-7 cellular clone (Huh7_HBI10A) containing a HCV-replicon and is based on the detection of the viral NS3 protein by ELISA. About 5000 Huh7_HBI10A cells (or Huh-7 cells as negative control), resuspended in 100 μL of complete DMEM were seeded into each well of a 96 well microtiter plate (Falcon sterile by Becton Dickinson). After 4 hours, 50 μL of an appropriate dissolution medium (e.g., DMEM) containing the compounds to test or DMSO (3%) were added, and incubated (37° C., 5% CO₂) for 4 days. The medium was removed by inversion and the cells were fixed by addition of 200 μL/well of ice-cold isopropanol 100%. After an incubation of 20 minutes at 4° C. the plate was washed with Washing Buffer 1 (=PBS 1×) and 300 μL of Buffer A (=PBS 1×, Triton ×100 0.1%, SDS 0.02%, 5% non-fat dry milk) was added in each well. The plates were incubated for 30 minutes at room temperature, and the blocking solution was removed by inversion. 100 μL of 1:2000 primary antibody (primary antibody=anti-NS3 mouse polyclonal 10E5/24) in Buffer A were added, and the plate was incubated for 120 minutes at room temperature. The plate was washed with solution Washing Buffer 2 (=PBS 1×, Triton ×100 0.1%, SDS 0.02%), followed by addition of 100 μL of 1:2000 secondary antibody AP-conjugated (anti-mouse IgG (Fc SPECIFIC) adsorbed with human IgG and rat serum proteins (SIGMA) or 1:4000 secondary antibody peroxidase-conjugated (anti-mouse IgG (Fc SPECIFIC)) adsorbed with bovine, horse and human serum proteins (SIGMA) in Buffer A. The plate was incubated for 120 minutes at room temperature and washed with Washing Buffer 2.

When secondary antibody AP-conjugated was used, 100 μL of AP substrate (SIGMA 104 Phosphatase substrate tablets; i.e., one tablet in 5 ml of AP buffer: diethanolamine solution 10%, pH 9.6-9.8.) were added to each well, and the plate was read in a conventional ELISA plate reader set at 405-620 nm.

When peroxidase-conjugated secondary was used, 100 μL of TMB substrate (SIGMA) were added to each well, and the plate was incubated for 20′ in the dark. 50 μL of 0.5M H₂SO₄ were then added, and the plate was read in a conventional ELISA plate reader set at 450-620 nm.

The specific compounds disclosed above (i.e., compounds 1 to 11) have all exhibited IC₅₀ values of less than about 50 μM in the RHEPLISA assay.

### EXAMPLE 2

### Cytoxicity Assay (SRB)

About 5000 Huh7_{—HBI}10A cells, resuspended in 100 μL of complete DMEM were seeded into each well of a 96 well microtiter plate (Falcon sterile by Becton Dickinson) and incubated (37° C., 5% CO₂) for 24 hours. The medium was removed and the cells were fixed by incubation with 50 μL of 50% TCA for 1 hour at 4° C., 200 μL of ice-cold isopropanol 100% for 20 minutes at 4° C., or 100 μL of 10% paraformaldehyde for 20 minutes at room temperature. The plate was washed with water when either TCA or paraformaldehyde was used to fix the cells and with PBS1× when isopropanol was used to fix the cells. All washings were run 3 times. After drying the plate, 200 μL of SRB 1× solution (prepared fresh from a SRB solution 10×:4% sulforhodamine B (Sigma) in 10% acetic acid) was added to each well and incubated for 30 minutes at room temperature. The SRB 1× solution was removed by inversion and the plate was washed in 1% acetic acid 3 times. To each well was added 200 μL of 10 mM Tris pH 10.5, the plate was shaken till the color of the solution in the wells was uniform, and the plate was read in a conventional ELISA plate reader set at 570 nm.

The specific compounds disclosed above have all exhibited cytotoxicity in the foregoing assay of less than about 20 percent using a 2 μM dose of the compound. Each of the compounds exhibited anti-HCV activity in the RHEPLISA assay at concentrations well below a concentration that would be associated with significant cytotoxicity in the instant assay.

The Huh-7 cell line is well known and is described in, for example, H. Nakabayashi et al., Cancer Research 1982, 42: 3858-3863 and in EP 1,043,399.

HCV replication systems can be obtained using techniques such as those described in Lohmann et al., Science 1999, 285: 110-113 (hereinafter referred to as “Lohman et al. 1999”). The development of this system was based on an experimental strategy that allowed selection of cells capable of supporting HCV replication. Selection can be achieved by using bicistronic RNA replicons expressing a selectable marker, the neomycin phosphotransferase. Transfection of these replicons in the human hepatoma cell line Huh-7, followed by cultivation in the presence of neomycin sulfate (G418), permits the isolation of clones that support HCV replication.

Plasmids pHCVNeol17.wt was assembled by several subcloning steps and contains the cDNA coding for an HCV bicistronic replicon identical to replicon I₃₇₇neo/NS3-3′/wt described by Lohmann et al. 1999, under the control of a T7 promoter.

The nucleic acid sequence for the pHCVNeol7.wt coding strand (SEQ. ID. No.: 1) is as follows:

| | |
|---|---|
| gccagccccc gattgggggc gacactccac catagatcac tcccctgtga ggaactactg | 60 |
| tcttcacgca gaaagcgtct agccatggcg ttagtatgag tgtcgtgcag cctccaggac | 120 |
| cccccctccc gggagagcca tagtggtctg cggaaccggt gagtacaccg gaattgccag | 180 |
| gacgaccggg tcctttcttg gatcaacccg ctcaatgcct ggagatttgg gcgtgccccc | 240 |
| gcgagactgc tagccgagta gtgttgggtc gcgaaaggcc ttgtggtact gcctgatagg | 300 |
| gtgcttgcga gtgccccggg aggtctcgta gaccgtgcac catgagcacg aatcctaaac | 360 |
| ctcaaagaaa aaccaaaggg cgcgccatga ttgaacaaga tggattgcac gcaggttctc | 420 |
| cggccgcttg ggtggagagg ctattcggct atgactgggc acaacagaca atcggctgct | 480 |
| ctgatgccgc cgtgttccgg ctgtcagcgc aggggcgccc ggttcttttt gtcaagaccg | 540 |
| acctgtccgg tgccctgaat gaactgcagg acgaggcagc gcggctatcg tggctggcca | 600 |
| cgacgggcgt tccttgcgca gctgtgctcg acgttgtcac tgaagcggga agggactggc | 660 |
| tgctattggg cgaagtgccg gggcaggatc tcctgtcatc tcaccttgct cctgccgaga | 720 |
| aagtatccat catggctgat gcaatgcggc ggctgcatac gcttgatccg gctacctgcc | 780 |
| cattcgacca ccaagcgaaa catcgcatcg agcgagcacg tactcggatg gaagccggtc | 840 |
| ttgtcgatca ggatgatctg gacgaagagc atcaggggct cgcgccagcc gaactgttcg | 900 |
| ccaggctcaa ggcgcgcatg cccgacggcg aggatctcgt cgtgacccat ggcgatgcct | 960 |
| gcttgccgaa tatcatggtg gaaaatggcc gcttttctgg attcatcgac tgtggccggc | 1020 |
| tgggtgtggc ggaccgctat caggacatag cgttggctac ccgtgatatt gctgaagagc | 1080 |
| ttggcggcga atgggctgac cgcttcctcg tgctttacgg tatcgccgct cccgattcgc | 1140 |
| agcgcatcgc cttctatcgc cttcttgacg agttcttctg agtttaaaca gaccacaacg | 1200 |
| gtttccctct agcgggatca attccgcccc tctccctccc ccccccctaa cgttactggc | 1260 |
| cgaagccgct tggaataagg ccggtgtgcg tttgtctata tgttattttc caccatattg | 1320 |
| ccgtcttttg gcaatgtgag ggcccggaaa cctggccctg tcttcttgac gagcattcct | 1380 |
| aggggtcttt cccctctcgc caaaggaatg caaggtctgt tgaatgtcgt gaaggaagca | 1440 |
| gttcctctgg aagcttcttg aagacaaaca acgtctgtag cgaccctttg caggcagcgg | 1500 |
| aaccccccac ctggcgacag gtgcctctgc ggccaaaagc cacgtgtata agatacacct | 1560 |
| gcaaaggcgg cacaacccca gtgccacgtt gtgagttgga tagttgtgga aagagtcaaa | 1620 |
| tggctctcct caagcgtatt caacaagggg ctgaaggatg cccagaaggt accccattgt | 1680 |
| atgggatctg atctggggcc tcggtgcaca tgctttacat gtgtttagtc gaggttaaaa | 1740 |
| aacgtctagg ccccccgaac cacggggacg tggttttcct ttgaaaaaca cgataatacc | 1800 |
| atggcgccta ttacggccta ctcccaacag acgcgaggcc tacttggctg catcatcact | 1860 |
| agcctcacag gccgggacag gaaccaggtc gagggggagg tccaagtggt ctccaccgca | 1920 |
| acacaatctt tcctggcgac ctgcgtcaat ggcgtgtgtt ggactgtcta tcatggtgcc | 1980 |
| ggctcaaaga cccttgccgg cccaaagggc ccaatcaccc aaatgtacac caatgtggac | 2040 |
| caggacctcg tcggctggca agcgcccccc ggggcgcgtt ccttgacacc atgcacctgc | 2100 |
| ggcagctcgg acctttactt ggtcacgagg catgccgatg tcattccggt gcgccggcgg | 2160 |
| ggcgacagca gggggagcct actctccccc aggcccgtct cctacttgaa gggctcttcg | 2220 |
| ggcggtccac tgctctgccc ctcggggcac gctgtgggca tctttcgggc tgccgtgtgc | 2280 |
| acccgagggg ttgcgaaggc ggtggacttt gtacccgtcg agtctatgga aaccactatg | 2340 |
| cggtccccgg tottcacgga caactcgtcc cctccggccg taccgcagac attccaggtg | 2400 |
| gcccatctac acgcccctac tggtagcggc aagagcacta aggtgccggc tgcgtatgca | 2460 |
| gcccaagggt ataaggtgct tgtcctgaac ccgtccgtcg ccgccaccct aggtttcggg | 2520 |
| gcgtatatgt ctaaggcaca tggtatcgac cctaacatca gaaccggggt aaggaccatc | 2580 |
| accacgggtg cccccatcac gtactccacc tatggcaagt ttcttgccga cggtggttgc | 2640 |
| tctgggggcg cctatgacat cataatatgt gatgagtgcc actcaactga ctcgaccact | 2700 |
| atcctgggca tcggcacagt cctggaccaa gcggagacgg ctggagcgcg actcgtcgtg | 2760 |
| ctcgccaccg ctacgcctcc gggatcggtc accgtgccac atccaaacat cgaggaggtg | 2820 |
| gctctgtcca gcactggaga aatccccttt tatggcaaag ccatccccat cgagaccatc | 2880 |
| aaggggggga ggcacctcat tttctgccat tccaagaaga aatgtgatga gctcgccgcg | 2940 |
| aagctgtccg gcctcggact caatgctgta gcatattacc ggggccttga tgtatccgtc | 3000 |
| ataccaacta gcggagacgt cattgtcgta gcaacggacg ctctaatgac gggctttacc | 3060 |
| ggcgatttcg actcagtgat cgactgcaat acatgtgtca cccagacagt cgacttcagc | 3120 |
| ctggacccga ccttcaccat tgagacgacg accgtgccac aagacgcggt gtcacgctcg | 3180 |
| cagcggcgag gcaggactgg taggggcagg atgggcattt acaggtttgt gactccagga | 3240 |
| gaacggccct cgggcatgtt cgattcctcg gttctgtgcg agtgctatga cgcgggctgt | 3300 |
| gcttggtacg agctcacgcc cgccgagacc tcagttaggt tgcgggctta cctaaacaca | 3360 |
| ccagggttgc ccgtctgcca ggaccatctg gagttctggg agagcgtctt tacaggcctc | 3420 |
| acccacatag acgcccattt cttgtcccag actaagcagg caggagacaa cttcccctac | 3480 |
| ctggtagcat accaggctac ggtgtgcgcc agggctcagg ctccacctcc atcgtgggac | 3540 |
| caaatgtgga agtgtctcat acggctaaag cctacgctgc acgggccaac gcccctgctg | 3600 |
| tataggctgg gagccgttca aaacgaggtt actaccacac accccataac caaatacatc | 3660 |
| atggcatgca tgtcggctga cctggaggtc gtcacgagca cctgggtgct ggtaggcgga | 3720 |
| gtcctagcag ctctggccgc gtattgcctg acaacaggca gcgtggtcat tgtgggcagg | 3780 |
| atcatcttgt ccggaaagcc ggccatcatt cccgacaggg aagtccttta ccgggagttc | 3840 |
| gatgagatgg aagagtgcgc ctcacacctc ccttacatcg aacagggaat gcagctcgcc | 3900 |
| gaacaattca aacagaaggc aatcgggttg ctgcaaacag ccaccaagca agcggaggct | 3960 |
| gctgctcccg tggtggaatc caagtggcgg accctcgaag ccttctgggc gaagcatatg | 4020 |
| tggaatttca tcagcgggat acaatattta gcaggcttgt ccactctgcc tggcaacccc | 4080 |
| gcgatagcat cactgatggc attcacagcc tctatcacca gcccgctcac cacccaacat | 4140 |
| accctcctgt ttaacatcct ggggggatgg gtggccgccc aacttgctcc tcccagcgct | 4200 |
| gcttctgctt tcgtaggcgc cggcatcgct ggagcggctg ttggcagcat aggccttggg | 4260 |
| aaggtgcttg tggatatttt ggcaggttat ggagcagggg tggcaggcgc gctcgtggcc | 4320 |
| tttaaggtca tgagcggcga gatgccctcc accgaggacc tggttaacct actccctgct | 4380 |
| atcctctccc ctggcgccct agtcgtcggg gtcgtgtgcg cagcgatact gcgtcggcac | 4440 |
| gtgggcccag gggagggggc tgtgcagtgg atgaaccggc tgatagcgtt cgcttcgcgg | 4500 |
| ggtaaccacg tctcccccac gcactatgtg cctgagagcg acgctgcagc acgtgtcact | 4560 |
| cagatcctct ctagtcttac catcactcag ctgctgaaga ggcttcacca gtggatcaac | 4620 |
| gaggactgct ccacgccatg ctccggctcg tggctaagag atgtttggga ttggatatgc | 4680 |
| acggtgttga ctgatttcaa gacctggctc cagtccaagc tcctgccgcg attgccggga | 4740 |
| gtccccttct tctcatgtca acgtgggtac aagggagtct ggcggggcga cggcatcatg | 4800 |
| caaaccacct gcccatgtgg agcacagatc accggacatg tgaaaaacgg ttccatgagg | 4860 |
| atcgtggggc ctaggacctg tagtaacacg tggcatggaa cattccccat taacgcgtac | 4920 |
| accacgggcc cctgcacgcc ctccccggcg ccaaattatt ctagggcgct gtggcgggtg | 4980 |
| gctgctgagg agtacgtgga ggttacgcgg gtgggggatt tccactacgt gacgggcatg | 5040 |
| accactgaca acgtaaagtg cccgtgtcag gttccggccc ccgaattctt cacagaagtg | 5100 |
| gatggggtgc ggttgcacag gtacgctcca gcgtgcaaac ccctcctacg ggaggaggtc | 5160 |
| acattcctgg tcgggctcaa tcaatacctg gttgggteac agctcccatg cgagcccgaa | 5220 |
| ccggacgtag cagtgctcac ttccatgctc accgacccct cccacattac ggcggagacg | 5280 |
| gctaagcgta ggctggccag gggatctccc ccctccttgg ccagctcatc agctagccag | 5340 |
| ctgtctgcgc cttccttgaa ggcaacatgc actacccgtc atgactcccc ggacgctgac | 5400 |
| ctcatcgagg ccaacctcct gtggcggcag gagatgggcg ggaacatcac ccgcgtggag | 5460 |
| tcagaaaata aggtagtaat tttggactct ttcgagccgc tccaagcgga ggaggatgag | 5520 |
| agggaagtat ccgttccggc ggagatcctg cggaggtcca ggaaattccc tcgagcgatg | 5580 |
| cccatatggg cacgcccgga ttacaaccct ccactgttag agtcctggaa ggacccggac | 5640 |
| tacgtcccsc cagtggtaca cgggtgtcca ttgccgcctg ccaaggcccc tccgatacca | 5700 |
| cctccacgga ggaagaggac ggttgtcctg tcagaatcta ccgtgtcttc tgccttggcg | 5760 |
| gagctcgcca caaagacctt cggcagctcc gaatcgtcgg ccgtcgacag cggcacggca | 5820 |
| acggcctctc ctgaccagcc ctccgacgac ggcgacgcgg gatccgacgt tgagtcgtac | 5880 |
| tcctccatgc ccccccttga gggggagccg ggggatcccg atctcagcga cgggtcttgg | 5940 |
| tctaccgtaa gcgaggaggc tagtgaggac gtcgtctgct gctcgatgtc ctacacatgg | 6000 |
| acaggcgccc tgatcacgcc atgcgctgcg gaggaaacca agctgcccat caatgcactg | 6060 |
| agcaactctt tgctccgtca ccacaacttg gtctatgcta caacatctcg cagcgcaagc | 6120 |
| ctgcggcaga agaaggtcac ctttgacaga ctgcaggtcc tggacgacca ctaccgggac | 6180 |
| gtgctcaagg agatgaaggc gaaggcgtcc acagttaagg ctaaacttct atccgtggag | 6240 |
| gaagcctgta agctgacgcc cccacattcg gccagatcta aatttggcta tggggcaaag | 6300 |
| gacgtccgga acctatccag caaggccgtt aaccacatcc gctccgtgtg gaaggacttg | 6360 |
| ctggaagaca ctgagacacc aattgacacc accatcatgg caaaaaatga ggttttctgc | 6420 |
| gtccaaccag agaagggggg ccgcaagcca gctcgcctta tcgtattccc agatttgggg | 6480 |
| gttcgtgtgt gcgagaaaat ggccctttac gatgtggtct ccaccctccc tcaggccgtg | 6540 |
| atgggctctt catacggatt ccaatactct cctggacagc gggtcgagtt cctggtgaat | 6600 |
| gcctggaaag cgaagaaatg ccctatgggc ttegcatatg acacccgctg ttttgactca | 6660 |
| acggtcactg agaatgacat ccgtgttgag gagtcaatct accaatgttg tgacttggcc | 6720 |
| cccgaagcca gacaggccat aaggtcgctc acagagcggc tttacatcgg gggccccctg | 6780 |
| actaattcta aagggcagaa ctgcggctat cgccggtgcc gcgcgagcgg tgtactgacg | 6840 |
| accagctgcg gtaataccct cacatgttac ttgaaggccg ctgcggcctg tcgagctgcg | 6900 |
| aagctccagg actgcacgat gctcgtatgc ggagacgacc ttgtcgttat ctgtgaaagc | 6960 |
| gcggggaccc aagaggacga ggcgagccta cgggccttca cggaggctat gactagatac | 7020 |
| tctgcccccc ctggggaccc gcccaaacca gaatacgact tggagttgat aacatcatgc | 7080 |
| tcctccaatg tgtcagtcgc gcacgatgca tctggcaaaa gggtgtacta tctcacccgt | 7140 |
| gaccccacca ccccccttgc gcgggctgcg tgggagacag otagacacac tccagtcaat | 7200 |
| tcctggctag gcaacatcat catgtatgcg cccaccttgt gggcaaggat gatcctgatg | 7260 |
| actcatttct tctccatcct tctagctcag gaacaacttg aaaaagccct agattgtcag | 7320 |
| atctacgggg cctgttactc cattgagcca cttgacctac ctcagatcat tcaacgactc | 7380 |
| catggcctta gcgcattttc actccatagt tactctccag gtgagatcaa tagggtggct | 7440 |
| tcatgcctca ggaaacttgg ggtaccgccc ttgcgagtct ggagacatcg ggccagaagt | 7500 |
| gtccgcgcta ggctactgtc ccaggggggg agggctgcca cttgtggcaa gtacctcttc | 7560 |
| aactgggcag taaggaccaa gctcaaactc actccaatcc cggctgcgtc ccagttggat | 7620 |
| ttatccagct ggttcgttgc tggttacagc gggggagaca tatatcacag cctgtctcgt | 7680 |
| gcccgacccc gctggttcat gtggtgccta ctcctacttt ctgtaggggt aggcatctat | 7740 |
| ctactcccca accgatgaac ggggagctaa acactccagg ccaataggcc atcctgtttt | 7800 |
| tttttcctct ttttttcctt ttctttcctt tggtggctcc atcttagccc tagtcacggc | 7920 |
| tagctgtgaa aggtccgtga gccgcttgac tgcagagagt gctgatactg gcctctctgc | 7980 |
| agatcaagta cttctagaga attctagctt ggcgtaatca tggtcatagc tgtttcctgt | 8040 |
| gtgaaattgt tatcagctca caattccaca caacatacga gccggaagca taaagtgtaa | 8100 |
| agcctgggat gcctaatgag tgagctaact cacattagtt gcgttgcgct cactgcccgc | 8160 |
| tttccagtcg ggaaacctgt cgtgccagct ccattagtga atcgtccaac gcacggggag | 8220 |
| aggcggtttg cgtattgggc gcacttccgc ttcctcgctc actgactcgc tgcgctcgtt | 8280 |
| cgttcggctg cggcgagccg tatcagctca ctcaaaggcg gtaatacggt tatccacaga | 8340 |
| atcaggggat aacgcaggaa agaccatgtg agcaaaaggc cagcaaaagg ccaggaaccg | 8400 |
| taaaaaggcc gcgttgctgg cgtttttcca taggctccgc ccccctgacg agcatcacaa | 8460 |
| aaatcgacgc tcaagtcaga ggtggcgaaa cccgacagga ctataaagat accaggcgtt | 8520 |
| tccccctgga agctccctcg tgcgctctcc tgttccgacc ctgccgctta ccggatacct | 8580 |
| gtccgccttt ctcccttcgg gaagcgtggc gctttctcat agctcacgct gtaggtatct | 8640 |
| cagttcggtg taggtcgttc gctccaagct gggctgtgtg cacgaacccc ccgttcagcc | 8700 |
| cgaccgctgc gccttatccg gtaactatcg tcttgagtcc aacccggtaa gacacgactt | 8760 |
| atcgccactg gcagcagcca ctggtaacag gattagcaga gcgaggtatg taggcggtgc | 8820 |
| tacagagttc ttgaagtggt ggcctaacta cggctacact agaaggacag tatttggtat | 8880 |
| ctgcgctctg ctgaagccag ttaccttcgg aaaaagagtt ggtagctctt gatccggcaa | 8940 |
| acaaaccacc gctggtagcg gtggtttttt tgtttgcaag cagcagatta cgcgcagaaa | 9000 |
| aaaaggatct caagaagatc ctttgatctt ttctacgggg tctgacgctc agtggaacga | 9060 |
| aaactcacgt taagggattt tggtcatgag attatcaaaa aggatcttca cctagatcct | 9120 |
| tttaaattaa aaatgaagtt ttaaatcaat ctaaagtata tatgagtaaa cttggtctga | 9180 |
| cagttaccaa tgcttaatca gtgaggcacc tatctcagcg atctgtctat ttcgttcatc | 9240 |
| catagttgcc tgactccccg tcgtgtagat aactacgata cgggagggct taccatctgg | 9300 |
| ccccagtgct gcaatgatac cgcgagaacc acgctcaccc gcaccagatt tatcagcaat | 9360 |
| aaaccagcca gccggaagtg cgctgcggag aagtggtcct gcaactttat ccgcctccat | 9420 |
| ccagtctatt agttgttgcc gggaagctag agtaagtagt tcgccagtca gcagtttgcg | 9480 |
| taacgtcgtt gccatagcaa caggcatcgt ggtgtcacgc tcgtcgtttg gtatggcttc | 9540 |
| attcagctcc ggctcccaac gatcaaggcg agttacatga tcccccatgt tgtgcaaaaa | 9600 |
| agcggttagc tccttcggtc ctccgatcgt tgtcagaagt aagttggccg cagtgttatc | 9660 |
| actcatggtt atggcagcac tgcataattc tcttactgtc atgccatccg taagatgctt | 9720 |
| ttctgtgact ggtgagtact caaccaagtc attctgagaa tagtgtatgc ggcgaccgag | 9780 |
| ttgctcttgc ccggcgtcaa tacgggataa taccgcgcca catagcagaa ctttaaaagt | 9840 |
| gctcatcatt ggaaaacgtt cttcggggcg aaaactctca aggatcttac cgctgttgag | 9900 |
| atccagttcg atgtaaccca ctcgtgcacc caactgatct tcagcatctt ttactttcac | 9960 |
| cagcgtttct gggtgagcaa aaacaggaag gcaaaatgcc gcaaaaaagg gaataagggc | 10020 |
| gacacggaaa tgttgaatac tcatactctt cctttttcaa tattattgaa gcatttatca | 10080 |
| gggttattgt ctcatgagcg gatacatatt tgaatgtatt tagaaaaata aacaaatagg | 10140 |
| ggttccgcgc acatttcccc gaaaagtgcc acctgacgtc taagaaacca ttattaccat | 10200 |
| gacattaacc tataaaaata ggcgtatcac gaagcccttt cgtctagcgc gtttcggtga | 10260 |
| tgacggtgaa aacctctgac acttgcagct cccgcagacg gtcacagctt gtctgtaagc | 10320 |
| ggatgccggg agcaggcaag cccgtcaggg cgcgtcagtg ggtgttggcg ggtgtcgggg | 10380 |
| ctggcttaac tatgcggcat cagagcagat tgtactgaga gtacaccaga tgcggtgtga | 10440 |
| aataccgcac agatgcgtaa ggagaaaata ccgcatcagc ctccattcgc cattcagact | 10500 |
| ccgcaactgt tgggaagggc ggtcagtacg cgcttcttcg ctattacgcc aactggcgaa | 10560 |
| agggggatgt gctgcaaggc gattaagttg ggtaacgcca gggttttccc aatcacgacg | 10620 |
| ttgtaaaacg acagccaatg aattgaagct tattaattct agactgaagc ttttaatacg | 10680 |
| actcactata | |

The different regions of pHCVNeol7.wt are as follows:

1-341: HCV 5′ non-translated region, drives translation of the core-neo fusion protein;

342-1181: core-neo fusion protein, selectable marker;

1190-1800: Internal ribosome entry site of the encephalomyocarditis virus, drives translation of the HCV NS region;

1801-7758: HCV polyprotein from non-structural protein 3 to non-structural protein 5B;

1801-3696: Non-structural protein 3 (NS3), HCV NS3 protease/helicase;

3697-3858: Non-structural protein 4A (NS4A), NS3 protease cofactor;

3859-4641: Non-structural protein 4B (NS4B);

4642-5982: Non-structural protein 5A (NS5A);

5983-7755: Non-structural protein SB (NS5B); RNA-dependent RNA polymerase

7759-7989: HCV 3′ non-translated region; and

7990-10690: plasmid sequences comprising origin of replication, beta lactamase coding sequence, and T7 promoter.

Plasmid pHCVNeol7.wt was digested with the Scal endonuclease (New England Biolabs) and transcribed in vitro with the T7 Megascript kit (Ambion). Transcription mixtures were treated with DNase (0.2 U/mL) to completely remove template DNA, extracted and precipitated as described Lohmann et al. 1999, and resuspended with phosphate buffered saline.

RNA transfection using Huh-7 cells and selection of G418 resistant colonies was performed as described in Lohmann et al. 1999. Huh-7 cells were grown in DMEM (Gibco, BRL) supplemented with 10% FCS. The cells were passed twice a week 1 to 5, using 1× trypsin/EDTA (Gibco, BRL). Huh-7 cells were transfected with pHCVNeol7.wt and cultured in the presence of G418. Several G418 resistant colonies were isolated, expanded, and molecularly characterized. Analysis of nucleic acids by PCR/reverse transcription-PCR, Northern blot and metabolic labeling with ³H-uridine indicated that all clones contained replicon RNA but not replicon DNA, demonstrating that G418 resistance was due to replication of viral RNA genomes. Furthermore, western blot and immunoprecipitation experiments showed that these clones expressed all HCV proteins. Clones differed in terms of cell morphology and growth rate. Replicons RNA copy number (500-10000 molecules/cell) and viral protein expression also varied between different clones. Clone Huh7_HBI10A was chosen to develop the RHEPLISA assay because of its good growth rate and high average level of viral RNA.

While the foregoing specification teaches the principles of the present invention, with examples provided for the purpose of illustration, the practice of the invention encompasses all of the usual variations, adaptations and/or modifications that come within the scope of the following claims.

What is claimed is:

1. A method for treating infection by the hepatitis C virus, treating hepatitis C or a related condition, delaying the onset of hepatitis C or a related condition, preventing hepatitis C or a related condition, or inhibiting replication of the hepatitis C virus, which comprises administering to a subject in need thereof an effective amount of a compound of Formula (I): 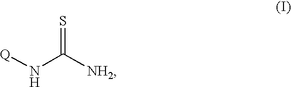
or a pharmaceutically acceptable salt thereof;
wherein Q is selected from the group consisting of: 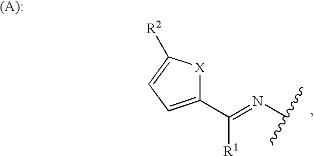
wherein
X is S or O;
R¹ is —H or —C₁₋₄ alkyl; and
R² is:
(1) —C₁₋₆ alkyl,
(2) —O—C₁₋₆ alkyl,
(3) —C₃₋₈ cycloalkyl,
(4) —O—C₃₋₈ cycloalkyl,
(5) —Si(R^{a})₃, in which one R^{a} group is phenyl optionally substituted with from 1 to 3 substituents each of which is independently —C₁₋₆ alkyl or —O—C₁₋₆ alkyl; and the other R^{a} groups are independently methyl, ethyl, methoxy, ethoxy, or phenyl optionally substituted with from 1 to 3 substituents each of which is independently —C₁₋₆ alkyl or —O—C₁₋₆ alkyl,
(6) —Cl or —Br,
(7) phenyl, optionally substituted with from 1 to 3 substituents each of which is independently —C₁₋₆ alkyl, —O—C₁₋₆ alkyl, or —C₃₋₈ cycloalkyl, or
(8) —(C₁₋₆ alkyl)-phenyl, in which the phenyl is optionally substituted with from 1 to 3 substituents each of which is independently —C₁₋₆ alkyl, —O—C₁₋₆ alkyl, or —C₃₋₈ cycloalkyl; 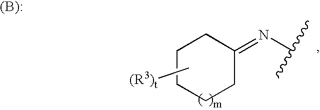
wherein
each R³ is independently a —C₁₋₆ alkyl group;
m is an integer from 2 to 9; and
t is zero, 1, 2, 3, or 4;
(C): 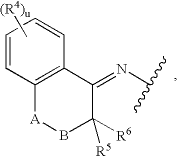
wherein
A is absent or —C(R^{b}R^{c})—;
B is —C(R^{d}R^{e})— or —NR^{f}—;
each R⁴ is independently:
(1) —C₁₋₆ alkyl,
(2) —O—C₁₋₆ alkyl,
(3) —C₃₋₈ cycloalkyl,
(4) —O—C₃₋₈ cycloalkyl,
(5) —Cl or —Br,
(6) phenyl, optionally substituted with from 1 to 3 substituents each of which is independently —C₁₋₆ alkyl, —O—C₁₋₆ alkyl, or —C₃₋₈ cycloalkyl, or
(7) —(C₁₋₆ alkyl)-phenyl, in which the phenyl is optionally substituted with from 1 to 3 substituents each of which is independently —C₁₋₆ alkyl, —O—C₁₋₆ alkyl, or —C₃₋₈ cycloalkyl;
R⁵ and R⁶ are each independently —H or —C₁₋₄ alkyl; or R⁵ and R⁶ taken together form oxo;
u is an integer equal to zero, 1, or 2;
R^{b} and R^{c} are each independently —H or —C₁₋₄ alkyl;
R^{d} and R^{e} are each independently —H or —C₁₋₄ alkyl; and
R^{f} is —H or —C₁₋₄ alkyl; 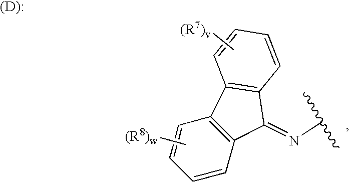
wherein each R⁷ and each R⁸ is independently:
(1) —C₁₋₆alkyl,
(2) —O—C₁₋₆ alkyl,
(3) —C₃₋₈ cycloalkyl,
(4) —O—C₃₋₈ cycloalkyl,
(5) —Cl or —Br,
(6) phenyl, optionally substituted with from 1 to 3 substituents each of which is independently —C₁₋₆ alkyl, —O—C₁₋₆ alky, or —C₃₋₈ cycloalkyl, or
(7) —(C₁₋₆ alkyl)-phenyl, in which the phenyl is optionally substituted with from 1 to 3 substituents each of which is independently —C₁₋₆ alkyl, —O—C₁₋₆ alkyl, or —C₃₋₈ cycloalkyl; and
v and w are each integers independently equal to zero, 1, 2 or 3; 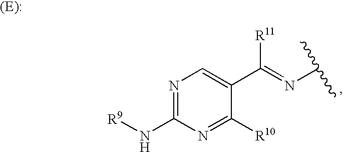
wherein R⁹ is:
(1) —C₁₋₆ alkyl,
(2) —C₃₋₈ cycloalkyl,
(3) phenyl, optionally substituted with from 1 to 3 substituents each of which is independently —C₁₋₆ alkyl, —O—C₁₋₆ alkyl, or —C₃₋₈ cycloalkyl, or
(4) —(C₁₋₆ alkyl)-phenyl, in which the phenyl is optionally substituted with from 1 to 3 substituents each of which is independently —C₁₋₆ alkyl, —O—C₁₋₆ alkyl, or —C₃₋₈ cycloalkyl;
R¹⁰ is —H or —C₁₋₄ alkyl; and
R¹¹ is —H or —C₁₋₄ alkyl; and 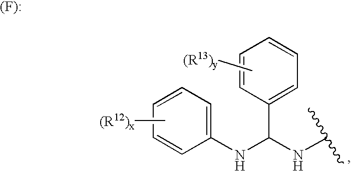
wherein each R¹² and each R¹³ is independently:
(1) —C₁₋₆ alkyl,
(2) —O—C₁₋₆ alkyl,
(3) —C₃₋₈ cycloalkyl,
(4) —O—C₃₋₈ cycloalkyl,
(5) —Cl or —Br,
(6) phenyl, optionally substituted with from 1 to 3 substituents each of which is independently —C₁₋₆ alkyl, —O—C₁₋₆ alkyl, or —C₃₋₈ cycloalkyl, or
(7) —(C₁₋₆ alkyl)-phenyl, in which the phenyl is optionally substituted with from 1 to 3 substituents each of which is independently —C₁₋₆ alkyl, —O—C₁₋₆ alkyl, or —C₃₋₈ cycloalkyl; and
x and y are each integers independently equal to zero, 1, 2 or 3.

2. The method according to claim 1, wherein the compound is a compound of Formula (II): 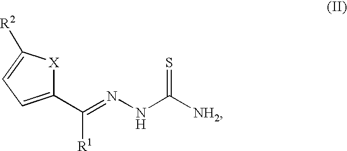
or a pharmaceutically acceptable salt thereof;
wherein X is S or O;
R¹ is —H, methyl, or ethyl; and
R² is:
(1) methyl,
(2) ethyl,
(3) methoxy,
(4) ethoxy,
(5) —C₅₋₆ cycloalkyl,
(6) —Si(R^{a})₃, in which one R^{a} group is phenyl and the other R^{a} groups are independently methyl, ethyl, or phenyl,
(7) —Cl,
(8) —Br,
(9) phenyl, or
(10) —CH2-phenyl.

3. The method according to claim 2, wherein in the compound of Formula (II) or a pharmaceutically acceptable salt thereof:
X is S or O;
R¹ is —H, methyl, or ethyl; and
R² is:
(1) methyl,
(2) ethyl,
(3) methoxy,
(4) ethoxy, 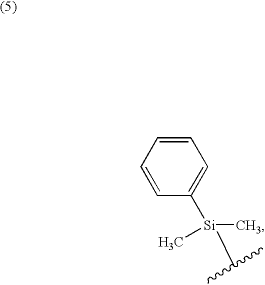
(6) —Cl, or
(7) —Br.

4. The method according to claim 3, wherein the compound is selected from the group consisting of: 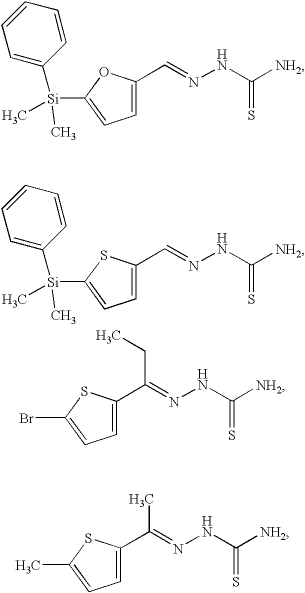
and pharmaceutically acceptable salts thereof.

5. The method according to claim 1, wherein the compound is a compound of Formula (III): 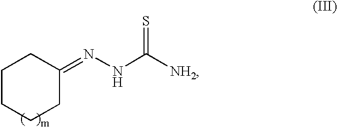
wherein m is an integer from 5 to 9.
or a pharmaceutically acceptable salt thereof.

6. The method according to claim 5, wherein the compound is 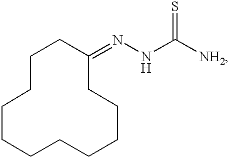
or a pharmaceutically acceptable salt thereof.

7. The method according to claim 1, wherein the compound is a compound of Formula (IV): 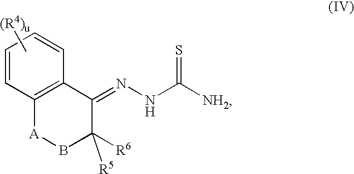
or a pharmaceutically acceptable salt thereof;
wherein:
A is absent or —CH₂—;
B is —CH₂— or —NR^{f}—, with the proviso that when B is —NR^{f}—, A is absent;
each R⁴ is independently:
(1) —C₁₋₄ alkyl,
(2) —O—C₁₋₄ alkyl, or
(3) —Cl or —Br,
R⁵ and R⁶ are both —H; or R⁵ and R⁶ taken together form oxo;
u is zero, 1, or 2; and
R^{f} is —H or methyl.

8. The method according to claim 7, wherein the compound is selected from the group consisting of: 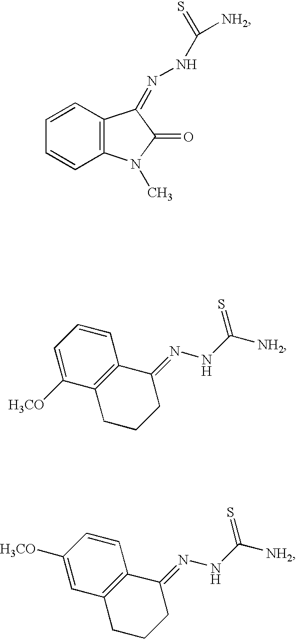
and pharmaceutically acceptable salts thereof.

9. The method according to claim 1, wherein the compound is a compound of Formula (V): 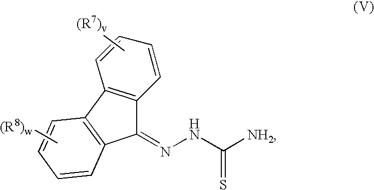
or a pharmaceutically acceptable salt thereof;
wherein:
each R⁷ and each R⁸ is independently:
(1) —C₁₋₄ alkyl,
(2) —O—C₁₋₄ alkyl, or
(3) —Cl or —Br; and
v and w are each integers independently equal to zero, 1, or 2.

10. The method according to claim 9, wherein the compound is 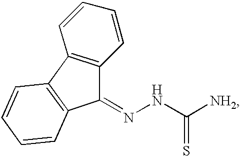
or a pharmaceutically acceptable salt thereof.

11. The method according to claim 1, wherein the compound is a compound of Formula (VI): 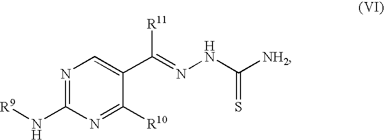
or a pharmaceutically acceptable salt thereof;
wherein R⁹ is —C₆₋₈ cycloalkyl;
R¹⁰ is —H or methyl; and
R¹¹ is —H or methyl.

12. The method according to claim 11, wherein the compound is 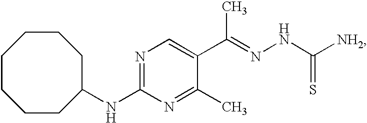
or a pharmaceutically acceptable salt thereof.

13. The method according to claim 1, wherein the compound is a compound of Formula (VII): 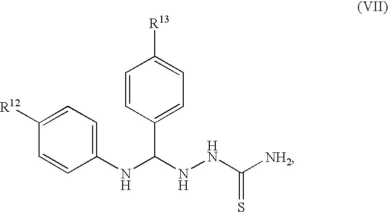
or a pharmaceutically acceptable salt thereof,
wherein R¹² and R¹³ are each independently:
(1) —H,
(2) —C₁₋₆ alkyl,
(3) —O—C₁₋₆ alkyl,
(4) —C₅₋₆ cycloalkyl,
(5) —O—C₅₋₆ cycloalkyl,
(6) —Cl or —Br,
(7) phenyl, optionally substituted with from 1 to 3 substituents each of which is independently —C₁₋₆ alkyl or —O—C₁₋₆ alkyl, or
(8) —(C₁₋₆ alkyl)-phenyl, in which the phenyl is optionally substituted with from 1 to 3 substituents each of which is independently —C₁₋₆ alkyl or —O—C₁₋₆ alkyl.

14. The method according to claim 13, wherein the compound is 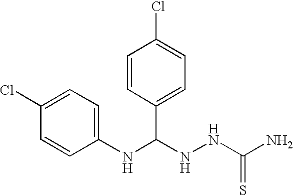
or a pharmaceutically acceptable salt thereof.

15. The method according to claim 1, which is a method for treating infection by the hepatitis C virus.

16. The method according to claim 1, which is a method for treating hepatitis C.

17. The method according to claim 1, which is a method for delaying the onset of hepatitis C.

18. The method according to claim 1, which is a method for preventing hepatitis C.

19. The method according to claim 1, which is a method for inhibiting replication of the hepatitis C virus.


SEQUENCE LISTING (1 sequence)

SEQ ID NO: 1 - DNA, 10690 bp, Artificial Sequence
  Plasmid
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaagggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatggaaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaaccggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcatcagctagccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagtacttctagagaattctagcttggcgtaatcatggtcatagctgtttcctgtgtgaaattgttatcagctcacaattccacacaacatacgagccggaagcataaagtgtaaagcctgggatgcctaatgagtgagctaactcacattagttgcgttgcgctcactgcccgctttccagtcgggaaacctgtcgtgccagctccattagtgaatcgtccaacgcacggggagaggcggtttgcgtattgggcgcacttccgcttcctcgctcactgactcgctgcgctcgttcgttcggctgcggcgagccgtatcagctcactcaaaggcggtaatacggttatccacagaatcaggggataacgcaggaaagaccatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcacccgcaccagatttatcagcaataaaccagccagccggaagtgcgctgcggagaagtggtcctgcaactttatccgcctccatccagtctattagttgttgccgggaagctagagtaagtagttcgccagtcagcagtttgcgtaacgtcgttgccatagcaacaggcatcgtggtgtcacgctcgtcgtttggtatggcttcattcagctccggctcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattaccatgacattaacctataaaaataggcgtatcacgaagccctttcgtctagcgcgtttcggtgatgacggtgaaaacctctgacacttgcagctcccgcagacggtcacagcttgtctgtaagcggatgccgggagcaggcaagcccgtcagggcgcgtcagtgggtgttggcgggtgtcggggctggcttaactatgcggcatcagagcagattgtactgagagtacaccagatgcggtgtgaaataccgcacagatgcgtaaggagaaaataccgcatcagcctccattcgccattcagactccgcaactgttgggaagggcggtcagtacgcgcttcttcgctattacgccaactggcgaaagggggatgtgctgcaaggcgattaagttgggtaacgccagggttttcccaatcacgacgttgtaaaacgacagccaatgaattgaagcttattaattctagactgaagcttttaatacgactcactata